(19) United States
(12) Patent Application Publication
(10) Pub. No.: US 2023/0035491 A1
(43) Pub. Date: Feb. 2, 2023

(54) METHODS, KITS AND COMPOSITIONS FOR REDUCING CARDIOTOXICITY ASSOCIATED WITH CANCER THERAPIES

(71) Applicant: Baruch S. Blumberg Institute, Doylestown, PA (US)

(72) Inventors: Richard G. Pestell, Fort Lauderdale, FL (US); Anthony Wayne Ashton, Ardmore, PA (US)

(21) Appl. No.: 17/785,385

(22) PCT Filed: Dec. 15, 2020
(86) PCT No.: PCT/US2020/065128

§ 371 Date: Jun. 14, 2022

Related U.S. Application Data
(60) Provisional application No. 62/948,301, filed on Dec. 15, 2019.

(51) Int. Cl. A61K 31/704; A61K 39/395; A61K 31/136; A61K 31/506; A61K 31/4706; A61K 31/517; A61K 31/439; A61P 35/00

(52) U.S. Cl. CPC A61K 31/704; A61K 39/3955; A61K 31/136; A61K 31/506; A61K 31/4706; A61K 31/517; A61K 31/439; A61P 35/00

(57) ABSTRACT

Described herein are methods for administering a chemotherapeutic agent to a patient in need thereof comprising administering an effective amount of a CCR5 antagonist contemporaneously with an effective amount of a chemotherapeutic agent. Also described are kits and compositions useful to implement the methods.

### CROSS-REFERENCE TO RELATED APPLICATION

This application claims the benefit of priority from U.S. Provisional Application No. 62/948,301, filed Dec. 15, 2019, the contents of which are hereby incorporated herein by reference in their entirety.

### BACKGROUND

The enhanced survival of cancer patients (>30% survival 5 years beyond initial diagnosis), is due in part to the use of chemotherapy and radiation. Unfortunately, chemotherapy and radiation are often associated with cardiotoxicity. For example, while anthracycline chemotherapy maintains a prominent role in treating many forms of cancer, cardiotoxic side effects limit their dosing as anthracycline-induced cardiotoxicity is cumulative and dose-dependent. In order to reduce cardiotoxicity, certain chemotherapeutic agents (e.g. anthracycline) have been formulated into liposomes to enhance penetration into leaking microvasculature found in tumors. However, despite these formulation improvements, nine percent (9%) of patients show diminished ejection fraction from the left ventricle within one (1) year of anthracycline therapy, increasing to >twenty-five percent (25%) of patients over five (5) years. Dexrazoxane is currently the only U.S. FDA-approved drug used clinically to prevent doxorubicin-induced (DOX-induced) cardiomyopathy. Its use has been limited for patients with metastatic breast cancer who have received a cumulative lifetime dose of at least 300 mg/m² of DOX, or an equivalent dose of other anthracyclines. However, dexrazoxane may reduce the efficacy of anthracycline, and increase the risk of myelotoxicity, and is therefore not used routinely.

Accordingly, there remains a critical need for methods, kits and compositions that are able to effectively deliver chemotherapeutic agents, without increasing the risk for chemotherapeutic induced side effects, such as cardio- or myelotoxicity. Embodiments of the present invention are designed to meet these and other needs.

### SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

In some embodiments, the present invention provides a method for administering a chemotherapeutic agent to a patient in need thereof comprising administering an effective amount of a CCR5 antagonist followed by administering an effective amount of a chemotherapeutic agent.

In other embodiments, the present invention provides a method of treating, preventing, or ameliorating a symptom associated with, the cardiotoxicity resulting from the administration of a chemotherapeutic agent comprising administering an effective amount of a CCR5 antagonist followed by administering an effective amount of a chemotherapeutic agent.

Still further embodiments of the present invention provide a method of enhancing cardiac function in a patient in need thereof comprising administering an effective amount of a CCR5 antagonist followed by administering an effective amount of a chemotherapeutic agent.

While other embodiments of the present invention provide a method of increasing survival rate or extending survival time in a patient undergoing treatment with a chemotherapeutic agent comprising administering an effective amount of a CCR5 antagonist followed by administering an effective amount of a chemotherapeutic agent.

Some embodiments of the present invention provide a method for reducing the effective dose of a chemotherapeutic agent in a patient in need thereof comprising administering an effective amount of a CCR5 antagonist followed by administering an effective amount of a chemotherapeutic agent.

As used herein, the term “contemporaneously administered” or “contemporaneous administration” is intended to include the administration of two therapeutic agents in a time frame, or a period of time, that is prior to, at about the same time, or shortly after

Certain embodiments of the present invention provide a method for reducing the cardiotoxicity associated with a chemotherapy, comprising co-administering an effective amount of a CCR5 antagonist and a chemotherapeutic agent. In some embodiments, the CCR5 antagonist and chemotherapeutic agent are administered contemporaneously. In some embodiments, the CCR5 antagonist is administered prior to the chemotherapeutic agent. In some embodiments, the CCR5 antagonist is administered from about 1 minute to about 72 hours prior to administration of the chemotherapeutic agent, optionally about 15 minutes, or 30 minutes, or 60 minutes, 90 minutes, or 2 hours, or 4 hours, or 8 hours, or 12 hours, or 18 hours, or 24 hours, or 36 hours, or 48 hours, or 60 hours or 72 hours, prior to administration of the chemotherapeutic agent. In some embodiments, the method further comprises the step of administering an additional dose of a CCR5 antagonist following administration of the chemotherapeutic agent.

Further areas of applicability of the present invention will become apparent from the detailed description provided hereinafter. It should be understood that the detailed description and specific examples, while indicating the typical embodiments of the invention, are intended for purposes of illustration only and are not intended to limit the scope of the invention.

### BRIEF DESCRIPTION OF THE DRAWINGS

The detailed description of the invention will be better understood when read in conjunction with the appended drawings. It should be understood, however, that the invention is not limited to the precise arrangements and instrumentalities of the embodiments shown in the drawings.

FIG. 1A depicts a quantitative immunofluorescence image of tumor tissue from node negative breast cancer patients. FIG. 1B depicts photos of photon flux imaging from breast tumors in nude mice. FIG. 1C depicts bioimaging of BCa lung metastasis in mice.

FIG. 2A depicts a graph comparing overall survival versus time for breast cancer patients with respect to CCR5 expression. FIG. 2B depicts a chart showing the result of gene expression from CCR5+ and CCR5− SUM-159 breast cancer cells.

FIG. 3 depicts a chart showing the fold change in gene expression within the human heart of either doxorubicin treated or donor controls.

FIGS. 4A to 4F depict images showing myocardial protein abundance of the CCR5-CCL5/CCL3 axis in heart tissues of either normal donors or patients with doxorubicin-induced cardiomyopathy (DoxTox).

FIG. 5A depicts a chart showing the ejection fraction over time for mice treated with a regimen of DOX. FIG. 5B depicts a chart showing end diastolic dimension over time for mice treated with a regimen of DOX. FIG. 5C depicts a chart showing end systolic volume over time for mice treated with a regimen of DOX. FIG. 5D depicts a chart showing the posterior wall thickness at the end of systole over time for mice treated with a regimen of DOX. FIG. 5E depicts the dosing protocol used.

FIG. 6A depicts images showing gene expression of heart tissue from mice given either saline or a chronic regimen of DOX. FIG. 6B depicts a chart showing mRNA expression of CCR5 and its ligands in the myocardium of mice treated with either saline (n=6), acute (n=18), or chronic (n=5) regimens of DOX.

FIG. 7 depicts a chart showing gene expression change within rat hearts in response to DOX treatment.

FIG. 8A depicts a graph showing CCR5 expression from MDA-MB-231 breast cancer cells. FIG. 8B depicts a graph showing CCR5 expression from progenitor induced pluripotent stem cells (iPSC).

FIG. 9 depicts a chart showing the percent of parental BCa cells expressing CCR5 under control or DOX regimen conditions.

FIG. 10A depicts a graph showing cell count compared to FL2 area. FIG. 10B depicts a chart showing cell apoptosis as a function of DOX concentration. FIG. 10C depicts a chart showing CCR5+ cell count compared to DOX concentration.

FIG. 11A depicts a graph showing the percent cell death compared to control of iPSC cells treated with either maraviroc or maraviroc and DOX. FIG. 11B depicts a graph showing MDA-MB-231 cell viability when treated with veliparib with either DMSO or maravoric.

FIG. 12A depicts a chart showing wall thickness of the left ventricular free wall thickness of mice compared to the left ventricular free wall (LVFW) and posterior wall (LVPW) at the end of either cardiac contraction (systole) or relaxation (diastole). FIG. 12B depicts a chart showing cardiac function with respect to stroke volume, ejection fraction, fractional shortening, and cardiac output.

FIG. 13 depicts a chart showing change in luciferase activity for various cells treated with DOX alone or with either maravoric or ranolazine.

FIG. 14 depicts a chart showing luciferase activity as a percent of vehicle for various cells treated with DOX alone or with either maravoric or ranolazine.

FIG. 15 depicts a chart showing the percent of apoptotic cells from various treatments including maraviroc and doxorubicin.

FIG. 16 depicts images showing various apoptotic mediators in mice hearts treated with vehicle or chronic maraviroc.

FIG. 17 depicts a chart showing the percent of apoptotic cells from various treatments including ranolazine and doxorubicin.

FIG. 18 depicts charts showing effects on cell proliferation from treatment with either ranolazine or ranolazine with doxorubicin.

FIG. 19 depicts a chart showing the probability of survival for mice treated with either doxorubicin or doxorubicin with maraviroc.

FIG. 20A depicts a diagram representing a protocol used for DOX testing in mice FIG. 20B depicts echocardiograms made at 8 weeks after the last DOX injection. FIG. 20c depicts charts showing changes to the heart in response to treatments with DOX alone or with Maraviroc.

FIG. 21 depicts a model by which dual purpose agents provide both cardio-protection and enhanced cancer cell killing.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight of the total composition. Reference to a molecule, or to molecules, being present at a “wt. %” refers to the amount of that molecule, or molecules, present in the composition based on the total weight of the composition.

According to the present application, use of the term “about” in conjunction with a numeral value refers to a value that may be +/−5% of that numeral. As used herein, the term “substantially free” is intended to mean an amount less than about 5.0 weight %, less than 3.0 weight %, 1.0 wt. %; preferably less than about 0.5 wt. %, and more preferably less than about 0.25 wt. % of the composition.

As used herein, the term “effective amount” refers to an amount that is effective to elicit the desired biological response, including the amount of a composition that, when administered to a subject, is sufficient to achieve an effect toward the desired result. The effective amount may vary depending on the composition, the disease, and its severity and the age, weight, etc., of the subject to be treated. The effective amount can include a range of amounts. As is understood in the art, an effective amount may be in one or more doses, i.e., a single dose or multiple doses may be required to achieve the desired endpoint.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

The present disclosure is directed toward compositions, kits and methods for reducing symptoms, such as cardiotoxicity and/or myelotoxicity, associated with chemotherapy use. In certain embodiments, the present disclosure is directed towards a method for administering a chemotherapeutic agent to a patient in need thereof. In other embodiments, the present disclosure is directed towards a method of treating, preventing, or ameliorating a symptom associated with cardiotoxicity resulting from administration of a chemotherapeutic agent. In other embodiments, the present disclosure is directed towards a method of enhancing cardiac function in a patient in need thereof. In other embodiments, the present disclosure is directed towards a method of increasing survival rate or extending survival time in a patient undergoing treatment with a chemotherapeutic agent. In other embodiments, the present disclosure is directed towards a method of reducing the effective dose of a chemotherapeutic agent in a patient in need thereof. In other embodiments, the present disclosure is directed towards a method for reducing the cardiotoxicity associated with a chemotherapy. In certain embodiments, the chemotherapeutic agent is a DNA damage inducing agent.

The present inventors have found that the G-protein coupled receptor CCR5 is expressed in ˜50% of human breast cancer (BCa) cells, and >95% of triple negative BCa cells, where it activates DNA repair and promotes metastasis. The present inventors have surprisingly and unexpectedly discovered that administering an effective amount of a G-protein-coupled receptor, C—C chemokine receptor type 5 (CCR5) antagonist in addition to administering an effective amount of a chemotherapeutic agent, provides for enhanced health benefit. Such enhanced health benefit may be exemplified by numerous aspects. In a first aspect, the health benefit may be to avoid increasing cardiotoxicity associated with administration of a chemotherapy. In another aspect, the health benefit may be to avoid increasing myelotoxicity associated with administration of a chemotherapy. In another aspect, the health benefit may be to reduce cardiotoxicity and/or myelotoxicity while concurrently providing an effective amount of a chemotherapeutic agent.

In certain embodiments, the CCR5 antagonist is selected from a small molecule; an immunotherapy; siRNA/CRISPR; a gene therapy; and a combination of two or more thereof. CCR5 antagonists are known in the art (See, for example, Kim et al., Expert Opin Investig Drugs, 2016, 25(12), 1377-1392; Thompson M A, Curr Opin HIV AIDS, 2018, 13(4), 346-53; Gu et al., Eur J Clin Microbiol Infect Dis., 2014, 33(11), 1881-7). In certain embodiments, the small molecule is selected from: maraviroc; vicriviroc; and a combination thereof.

The amount or concentration of CCR5 antagonist may vary. In certain embodiments, the effective amount of the CCR5 antagonist is from about 1 mg/kg/day to about 200 mg/kg/day, optionally from about 10 mg/kg/day to about 190 mg/kg/day, or about 20 mg/kg/day to about 180 mg/kg/day, or about 30 mg/kg/day to about 170 mg/kg/day, or about 40 mg/kg/day to about 160 mg/kg/day, or about 50 mg/kg/day to about 150 mg/kg/day, or about 60 mg/kg/day to about 140 mg/kg/day, or about 70 mg/kg/day to about 130 mg/kg/day, or about 80 mg/kg/day to about 120 mg/kg/day, or about 90 mg/kg/day to about 110 mg/kg/day, or about 100 mg/kg/day.

In certain embodiments, ranolazine may be used to provide health benefits selected from one or more of enhancing chemotherapy induced (such as DOX induced) cancer cell killing, reducing metastatic burden caused by chemotherapy (such as DOX), and/or provide cardioprotection from chemotherapy (such as DOX). In certain embodiments, ranolazine in the form of ranolazine dihydrochloride may be used.

The present invention may be utilized with one or more chemotherapeutic agents. Various chemotherapeutic agents are well known in the art. In certain embodiments, the chemotherapeutic agent is selected from an anthracycline; a Her2 inhibitor; an immune checkpoint inhibitor; and a combination of two or more thereof. In certain embodiments, the anthracycline is selected from: daunorubicin; doxorubicin; epirubicin; idarubicin; valrubicin; mitoxantrone; and a combination of two or more thereof. In certain embodiments, the Her2 inhibitor is selected from trastuzumab; lapatinib; neratinib; pertuzumab; dacomitinib; and a combination of two or more thereof. In certain embodiments, the immune checkpoint inhibitor comprises a CTLA4/PD-1/PD-L1 selected from: cemiplimab; nivolumab; pembrolizumab; avelumab; durvalumab; atezolizumab; ipilimumab; and a combination of two or more thereof.

In one aspect, the present disclosure therefore provides a method for administering a chemotherapeutic agent to a patient in need thereof comprising administering an effective amount of a CCR5 antagonist followed by administering an effective amount of a chemotherapeutic agent. In further embodiments, the present disclosure provides for a method of treating, preventing, or ameliorating a symptom associated with cardiotoxicity resulting from the administration of a chemotherapeutic agent comprising administering an effective amount of a CCR5 antagonist followed by administering an effective amount of a chemotherapeutic agent. In other embodiments, the present disclosure provides for a method of enhancing cardiac function in a patient in need thereof comprising administering an effective amount of a CCR5 antagonist followed by administering an effective amount of a chemotherapeutic agent. In yet other embodiments, the present disclosure provides for a method of increasing survival rate or extending survival time in a patient undergoing treatment with a chemotherapeutic agent comprising administering an effective amount of a CCR5 antagonist followed by administering an effective amount of a chemotherapeutic agent. In yet other embodiments, the present disclosure provides for a method of reducing the effective dose of a chemotherapeutic agent in a patient in need thereof comprising administering an effective amount of a CCR5 antagonist followed by administering an effective amount of a chemotherapeutic agent. In other embodiments, the present disclosure provides for method for reducing the cardiotoxicity associated with a chemotherapy, comprising co-administering an effective amount of a CCR5 antagonist and a chemotherapeutic agent.

In certain embodiments, the CCR5 antagonist is administered prior to administration of the chemotherapeutic agent. In other embodiments, the CCR5 antagonist is co-administered with administration of the chemotherapeutic agent. In various embodiments, the CCR5 antagonist and chemotherapeutic agent are administered contemporaneously. In certain embodiments, the CCR5 antagonist is administered prior to the chemotherapeutic agent. In certain embodiments, the CCR5 antagonist may be administered from about 1 minute to about 72 hours prior to administration of the chemotherapeutic agent, optionally about 15 minutes, or 30 minutes, or 60 minutes, 90 minutes, or 2 hours, or 4 hours, or 8 hours, or 12 hours, or 18 hours, or 24 hours, or 36 hours, or 48 hours, or 60 hours or 72 hours, prior to administration of the chemotherapeutic agent. In further embodiments, in addition to a contemporaneous CCR5 antagonist and chemotherapeutic agent administration or a CCR5 antagonist administration prior to the chemotherapeutic agent administration, further step comprising administering an additional dose of a CCR5 antagonist following administration of the chemotherapeutic agent may be performed.

In one aspect, the present disclosure therefore provides for a composition comprising an effective amount of a CCR5 antagonist and an effective amount of a chemotherapeutic agent. In other embodiments, the present disclosure therefore provides for composition comprising an effective amount of doxorubicin; an effective amount of lapatinib and/or rapamycin; and a pharmaceutically acceptable carrier.

In another aspect, the present disclosure provides for A kit for reducing cardiotoxicity associated with chemotherapy comprising a CCR5 antagonist; a chemotherapeutic agent; and instructions for the administration of each.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

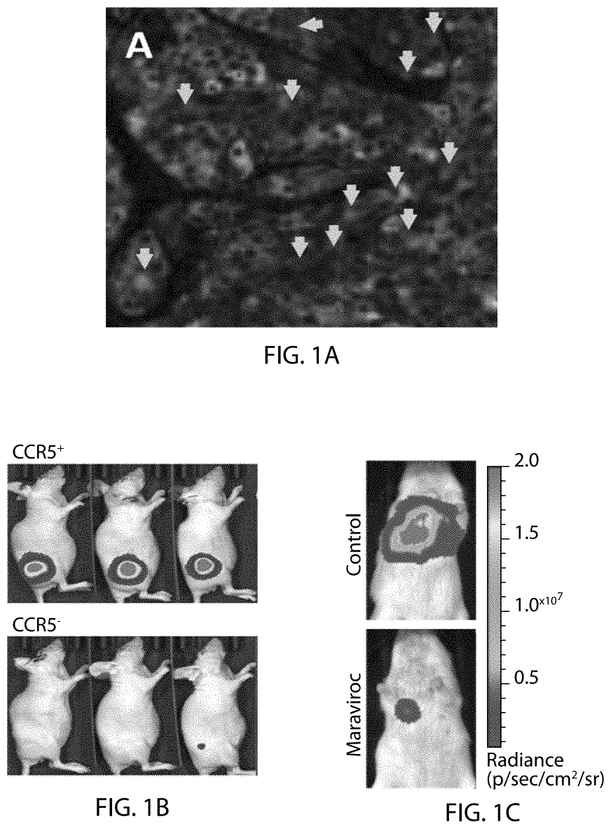

FIG. 1a shows quantitative immunofluorescence on tumor tissue from node-negative breast cancer patients. Antibodies used were against pan-cytokeratin (FITC labelled, yielding a green color) and CCR5 (Cy5 conjugated, yielding a red color, also shown by the arrows). After deparaffinization and rehydration, antigen retrieval was performed in citrate buffer (pH 9). After blocking sections were incubated with antibodies against CCR5 or pan-cytokeratin for 1 hr. Anti-CCR5 and anti-pan-cytokeratin binding was visualized using Cy5 and Alexa 555 conjugated secondary antibodies. DAPI was used for nuclear visualization. Slides were imaged on an Aperio Scanscope FL and expression quantified using Tissue Studio (Definiens) image analysis. The results show that CCR5 promotes breast cancer cell growth and metastasis.

FIG. 1b are photos of photon flux imaging from breast tumors in mice derived from injection of CCR5+ versus CCR5− luc2 stable SUM-159 breast cancer cells (n=5). Briefly, twelve week old female NCr nu/nu mice (NCI, Bethesda, Md.) received 4000 FACS sorted CCR5+ or CCR5− cells suspended in diluted Matrigel Basement Membrane Matrix by subcutaneous injection. Tumor progression was followed by measurement of bioluminescence once a week until tumor excision, using a IVIS LUMINA XR system (Caliper Life Sciences, Waltham, Mass.). To visualize bioluminescence, mice received an injection of d-Luciferin (15 mg/ml) and were imaged about fifteen minutes later.

FIG. 1c shows that BCa lung metastasis are reduced by CCR5 antagonists (n=6). SUM-159 cells expressing Luc2-eGFP were introduced by intracardiac injection into 8-week old female NOD/SCID mice at 2×10⁵ cells/mouse). Mice were treated immediately after injection by oral gavage with Maraviroc (8 mg/kg every 12 hr) or control/vehicle (5% DMSO in acidified water). Bioluminescence imaging was performed after intraperitoneal (i.p.) injection with 200 μL of D-luciferin at 30 mg/ml.

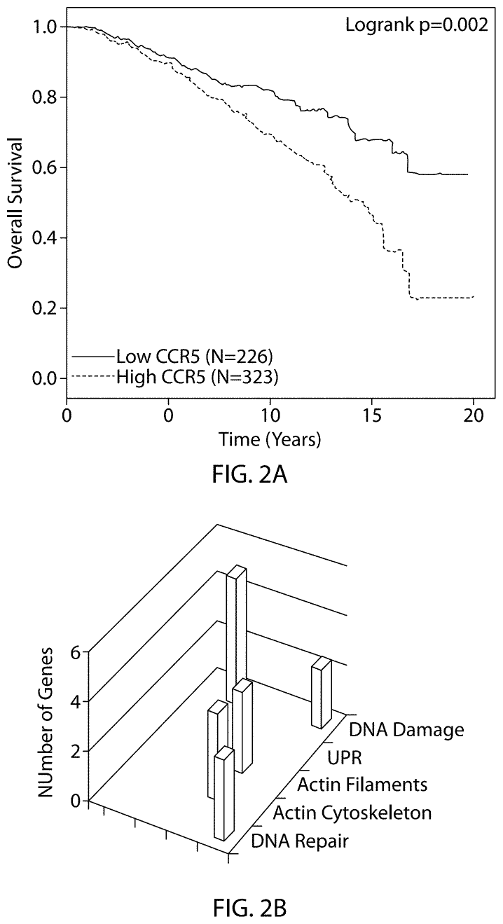

FIG. 2a shows that presence of CCR5 worsens patient survival through mediating drug resistance of tumor cells. Specifically, CCR5+ BCa correlates with poor prognosis. Based on CCR5 staining (as shown in FIG. 1a), patients were segregated into either high or low expression groups. Analysis of overall survival was conducted using Xtile to establish data-driven, optimal cutpoint for dichotomization (high vs. low) of CCR5 levels in the cohort. Kaplan-Meier plots of survival for high cytoplasmic CCR5 vs. low cytoplasmic CCR5 were prepared. SPSS software was used to evaluate the differences between patients with high vs. low CCR5 levels using the Kaplan-Meier estimator of the survival curves and log-rank test, and Cox regression was used for multivariable analyses.

FIG. 2b shows that CCR5 increases DNA repair mechanisms in BCa cells. Briefly, mRNA was isolated from CCR5+ and CCR5− SUM-159 breast cancer cells obtained by FACS sorting. Gene-ontology pathway analysis of the resulting microarray gene expression data indicated pathways involved with “response to DNA damage stimulus”, “DNA repair”, “response to unfolded proteins”, “actin filament based process” and “actin cytoskeleton organization” are elevated in CCR5+ BCa cells.

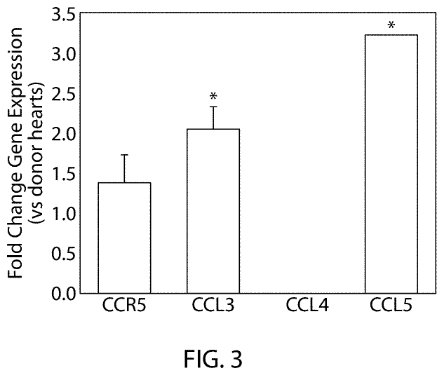

FIG. 3 shows mRNA microarray results (available from the Gene Expression Omnibus (GEO) database) exploring the basis for cardiotoxicity from doxorubicin within patients. The results show increased mRNA expression for CCR5 ligands CCL3 and CCL5, but not CCL4, in the heart of doxorubicin treated patients (n=7) but not donor controls (n=9). Data are shown as fold change to the donor group mean+/−SEM, * p<0.01.

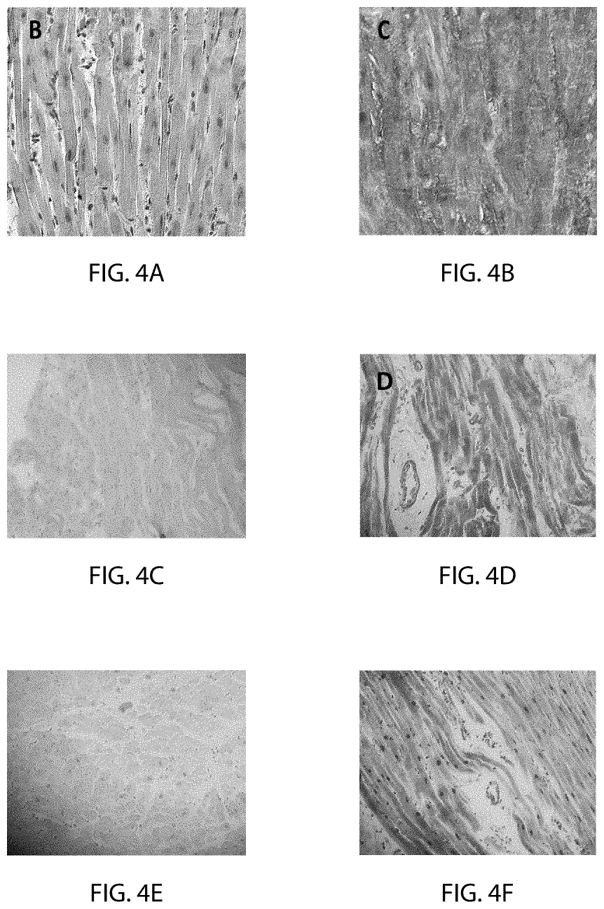

FIGS. 4a through 4f show myocardial biopsies from patients with either doxorubicin-induced cardiomyopathy or donor control heart. These images show that doxorubicin treatment increases the CCR5 signaling in the human heart. Samples were obtained from the Sydney heart bank. Biopsies were obtained at the time of transplant for the doxorubicin affected hearts. Average ejection fraction (EF) for doxorubicin affected hearts was 25-35% while for normal donor hearts was 65-70%, suggesting significant heart failure prior to explant. Heart biopsies were fixed in paraformaldehyde overnight prior to being embedded in paraffin blocks. Blocks were sectioned at 5 p.m. After deparaffinization and rehydration, antigen retrieval was performed in Tris buffer (pH 9) on the resulting sections. After blocking, sections were incubated with an antibody against CCR5, CCL3, or CCL5. Anti-CCR5 antibody binding was visualized using an HRP-conjugated second antibody and NOVA red substrate. Haematoxylin was used for nuclear visualization. CCR5, CCL3 and CCL5 expression were increased in myocardium affected by anthracycline cardiotoxicity (as shown in FIGS. 4b, 4d, and 4f; n=2) compared to normal donor heart tissue (FIGS. 4a, 4c, and 4e; n=2). While increased CCL3 and CCL5 expression match the increased mRNA expression in DOX treated patients, the discord between CCR5 protein and mRNA expression suggests a post-transcriptional mechanism of regulation.

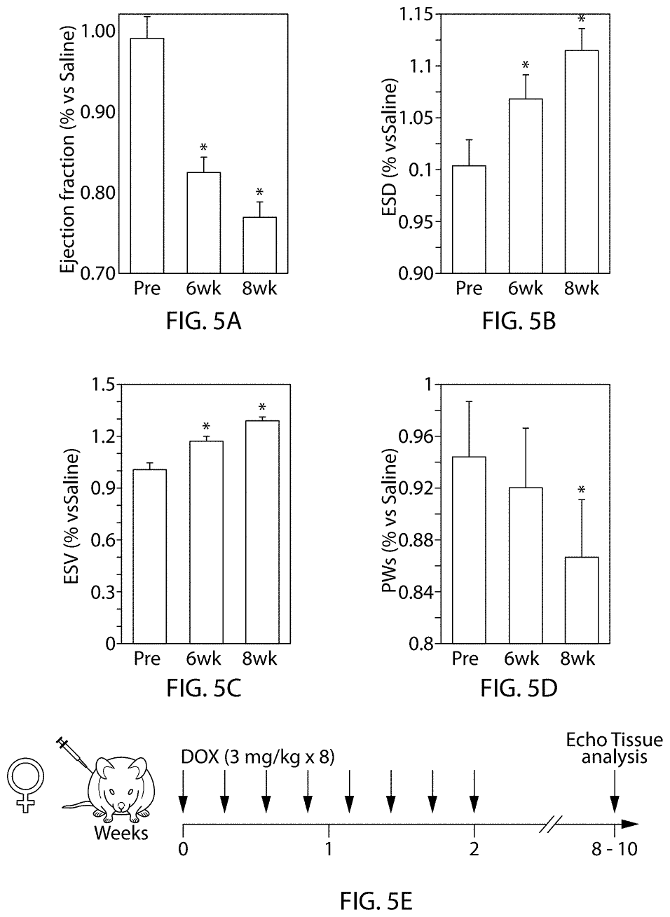

FIGS. 5a-5d show induction of stable mild heart failure in the chronic DOX model. Female mice (n=10) were treated with a chronic regimen of DOX (8×3 mg/kg over 2 weeks) and cardiac function assessed by echocardiography at 6 and 8 weeks after the first injections. Echocardiographic imaging was employed using the Vevo 2100 preclinical high-frequency ultrasound system (Visualsonics, Toronto, Canada). M-mode analysis of the resulting images provided indices of cardiac systolic and diastolic function. For FIG. 5a, the ejection fraction is the percentage of the left ventricular volume expelled with each cardiac contraction. For FIG. 5b, the ESD is the end diastolic dimension or the diameter of the heart at the end of relaxation (diastole). For FIG. 5c, the ESV is the end systolic volume or the volume of the heart at the end of contraction (systole). For FIG. 5d, the PWs is the posterior wall thickness at end of systole or the thickness of the myocardium at maximal ventricular contraction. All data are compared to the saline treated group and represent mean+/−SEM, p<0.01.

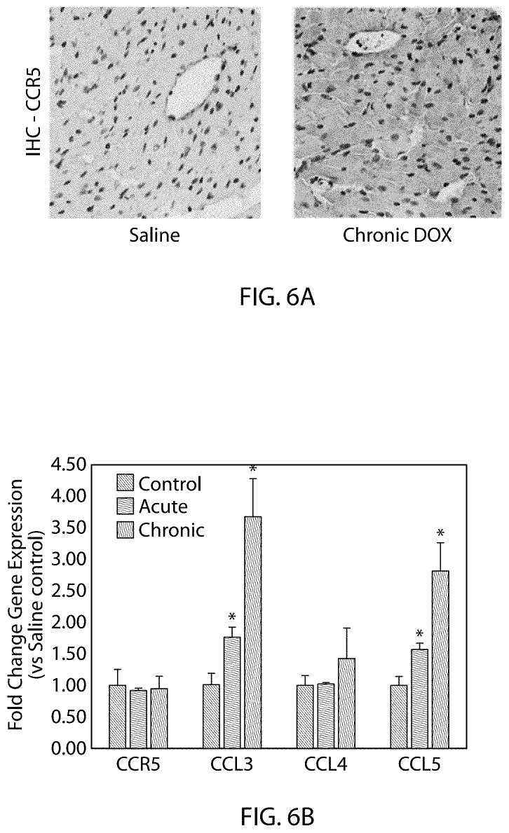

FIG. 6a shows that CCR5 expression is induced in the myocardium of mice by DOX. Mice were treated with a chronic regimen of DOX (8×3 mg/kg, IP, n=12) or saline (n=10), excised after 8 weeks and fixed in paraformaldehyde overnight before being embedded in paraffin blocks. Blocks were sectioned (5 μm) and, after deparaffinization and rehydration, antigen retrieval was performed in Tris buffer (pH 9). After blocking, sections were incubated with an antibody against CCR5. Anti-CCR5 antibody binding was visualized using an HRP-conjugated second antibody and Nova red substitute. Haematoxylin was used for nuclear visualization. CCR5 expression was increased in the myocardium of mice treated with doxorubicin (n=10) compared to saline controls (n=10).

FIG. 6b shows mRNA expression of CCR5 and its ligands (CCL3/MIP-la and CCL5/RANTES) in the myocardium of mice treated with either saline, chronic or acute regimens of DOX. mRNA microarray results (available from GEO database) exploring the basis for the cardiotoxicity of doxorubicin in mice and rats were obtained. Chronic exposure to DOX followed the regimen as shown in FIG. 5e. Acute exposure was a one-time IP injection of 15-20 mg/kg of doxorubicin. Control mice were injected with the same volume of saline, at the same frequency, at the same frequency, used for DOX treatment. Analysis of CCR5 expression showed increased mRNA expression for the CCR5 ligands CCL3 and CCL5, but not CCR5, in the heart of doxorubicin treated mice with changes in the chronic model more closely reproducing the phenotype of the human samples (see FIG. 3a). Data are represented as fold change to saline treated mice and shown as mean+/−SEM, p<0.01. As per human myocardium. the increased protein expression compared to mRNA expression suggests a post-transcriptional mechanism of regulation.

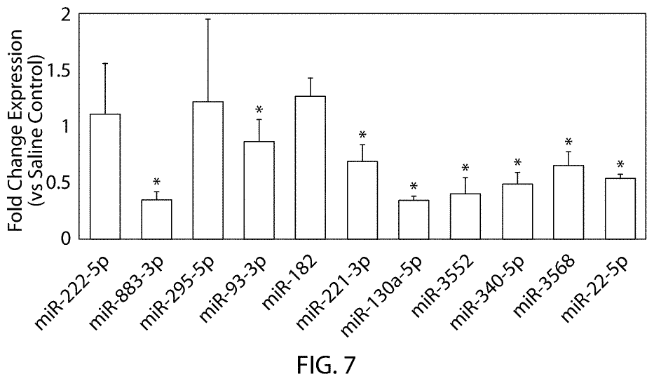

FIG. 7 shows that miRNAs capable of targeting CCR5 protein expression are lost from doxorubicin-treated myocardium. In silico analysis using the miRdb database (www.mirdb.org) identified a number of miRNAs that could potentially regulate translation of CCR5 in rats (see X-axis for identification of individual miRNAs). FIG. 7 shows the miRNA expression profiles from mRNA (including miRNA) isolated from the hearts of rats treated with an acute doxorubicin regimen (lx 20 mg/kg, IP, n=6) or control rats injected with the same volume of saline, at the same frequency, used for DOX treatment (n=6). miRNA expression profiles were generated using Illumina miRNA arrays. Analysis of miRNA arrays showed decreased expression of the majority of miRNAs targeting CCR5 (8/11 miRNAs) in the myocardium of DOX treated rats. Data rare represented as fold change to saline treated rats and shown as mean+/−SEM, * p<0.01.

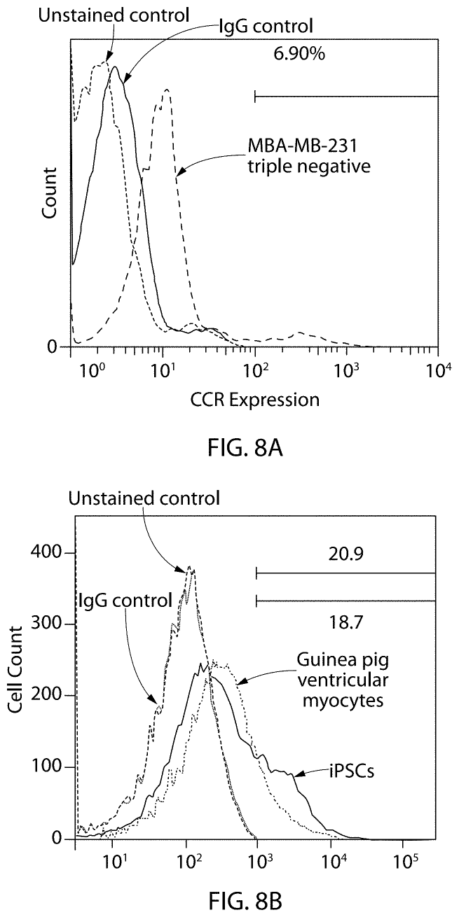

FIGS. 8a-b show CCR5 expression in cardiac progenitor and breast cancer cells. Visualized are flow cytometric histograms of CCR5 expression in MDA-MB-231 breast cancer cells (FIG. 8a) and cardiac progenitor iPSCs (FIG. 8b). Cells were grown in vitro under standard conditions for each cell line. Cells were harvested and pelleted by centrifugation. Cells were then suspended in PBS containing normal mouse IgG and rat anti-mouse Fcg III/II receptor antibody to block nonspecific binding. Cells were exposed allophycocyanin (APC)-labeled CCR5 antibody for 1 hour at 4° C. After washing, analysis of CCR5 expression on cell was conducted on FACSCalibur flow cytometer (BD Biosciences). The data was analyzed with FlowJo software (Tree Star, Inc.). CCR5 expression was observed on a subpopulation of MDA-MB-231 triple negative BCa cells in vitro when compared to controls unstained and IgG control. (FIG. 7a). Flow cytometry detection of CCR5 expression in iPSCs and guinea pig ventricular myocytes in vitro compared to controls unstained and IgG.

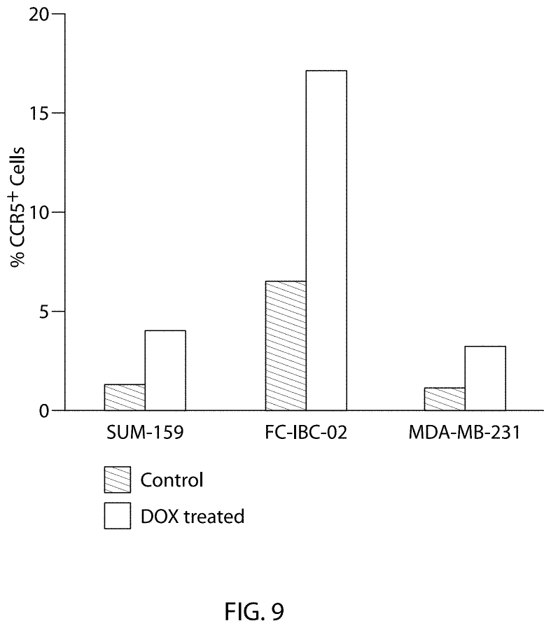

FIG. 9 shows doxorubicin treatment increases the CCR5+ expression in BCa cells. BCa cells were grown in vitro under standard conditions for control (“solid bar”) or DOX treatment (“non-solid bar”) for up to 80 days. SUM-159 cells were grown in 10 nmol/L doxorubicin for 1 month, then 20 nmol/L doxorubicin for 1 month, and then 40 nmol/L doxorubicin for 3 weeks, prior to analysis. FC-IBC-02 cells were grown in 40 nmol/L doxorubicin for 1 month prior to analysis. MDA-MB-231 cells were grown in 20 nmol/L doxorubicin for 1 month then 40 nmol/L doxorubicin for 3 weeks prior to analysis. At the conclusion of the 80 days, surviving cells were harvested and pelleted by centrifugation. Cells were suspended in PBS containing normal mouse IgG and rat anti-mouse Fcg III/II receptor antibody to block nonspecific binding. Cells were exposed allophycocyanin (APC)-labeled CCR5 antibody for 1 hour at 4° C. After washing, analysis of CCR5 expression on cell was conducted on FACSCalibur flow cytometer (BD Biosciences). The data was analyzed with FlowJo software (Tree Star, Inc.). DOX significantly increased CCR5 expression in all populations of treated BCa cells compared to untreated counterparts. Data are % cells expressing CCR5 (mean of n=3 determinations).

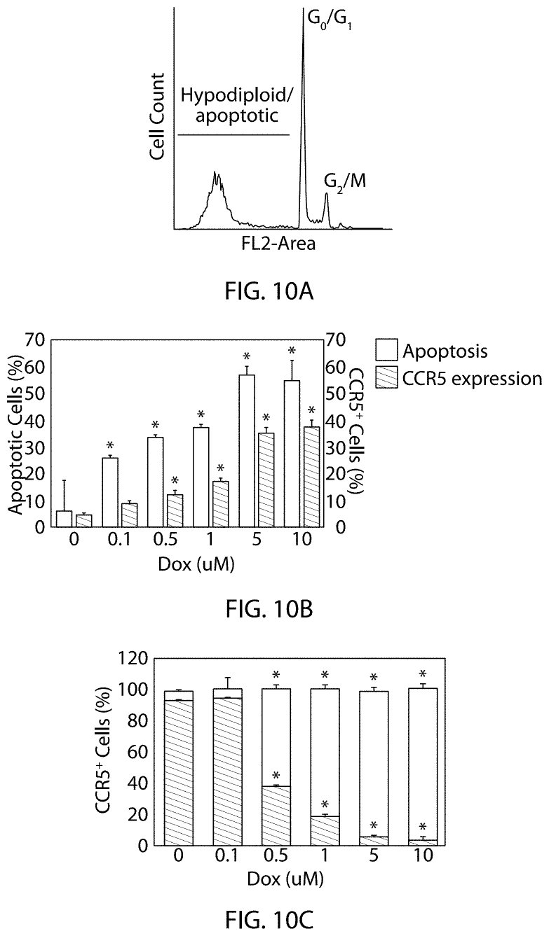

FIGS. 10a-c show CCR5+ cardiac progenitor iPSCs are sensitive to DOX killing. iPSC were plated at 2.5×10⁴ cells/cm² in vitro and cultured with varying DOX concentrations (0-10 m). After 24 hours, cells were harvested and pelleted by centrifugation. Some cells were suspended in PBS containing normal mouse IgG and rat anti-mouse Fcg III/II receptor antibody. Cells were exposed allophycocyanin (APC)-labeled CCR5 antibody for 1 hour at 4° C. After washing, analysis of CCR5 expression on cell was conducted on FACSCalibur flow cytometer (BD Biosciences). The data was analyzed with FlowJo software (Tree Star, Inc.). For cell death, cells were incubated with RNase A and propidium iodide to highlight DNA content and nuclear morphology. Cell death was identified by counting cells with nuclei that were smaller and with less DNA content compared to normal diploid cells (FIG. 10a). FIG. 10b shows that treatment of cardiac progenitor iPSCs with DOX promotes cell death in a dose dependent manner (n=5). Moreover, DOX increases CCR5 expression in cardiac progenitor iPSCs (n=3). As such, CCR5+ cardiac progenitor cells are more prone to DOX induced cell death from than their CCR5− counterparts. FIG. 10c shows an analysis of the iPSCs undergoing cell death indicates that the proportion of CCR5+ cardiac progenitor iPSCs is far greater than the CCR5-pool (n=3).

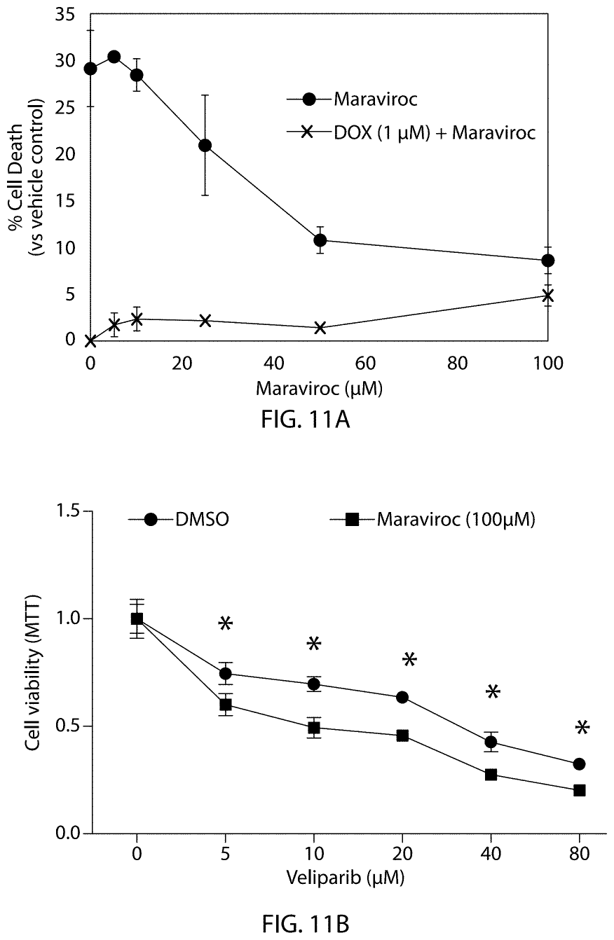

FIGS. 11a-b show that CCR5 inhibition by maraviroc promotes cardiac precursor survival and BCa cell killing. iPSC were plated at 2.5×10⁴ cells/cm² in vitro and cultured with saline or 1 μM DOX and varying concentrations of the CCR5 antagonist maraviroc (0-100 m). After 24 hours, cells were harvested and pelleted by centrifugation. For cell death, cells were incubated with RNase A and propidium iodide to highlight DNA content and nuclear morphology. Cell death was identified by counting cells with nuclei that were smaller and with less DNA content compared to normal diploid cells. Maraviroc alone had no significant effect on the viability of cardiac progenitor iPSCs but significantly, and dose dependently, rescues DOX induced cell death in cardiac precursor iPSCs. (mean+/−SD, n=3). FIG. 11b shows cell viability as a result of treatment with veliparib. MDA-MB-231 BCa cells were plated onto 96-well plates, allowed to adhere overnight, then treated with drug for 72 hours. Cells were incubated with DNA damaging agent (Veliparib) and either vehicle or maraviroc. A measurement of cell number was made at both the time of treatment (time 0) and after drug treatment (time 72) using CTG reagent (Promega, Madison, Wis.) to allow for calculation of percent growth inhibition and the dose required to inhibit growth rate by 50% (GR50). Maraviroc synergistically promotes BCa cell killing with veliparib. Data represents mean+/−SD (n=3).

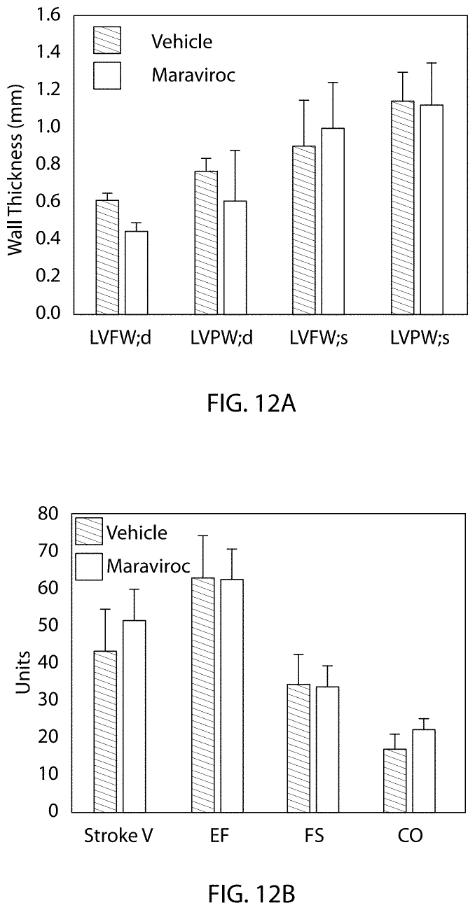

FIGS. 12a-b show that chronic consumption of CCR5 inhibitors do not affect murine cardiac function. Mice (n=3) were treated for 9 months with maraviroc (16 mg/kg, twice daily gavage) or vehicle. At the end of 9 months, measurements of cardiac function were made using echocardiography with the Vevo 770 preclinical high frequency ultrasound system (Visualsonics). M-mode analysis of the resulting images provided indices of cardiac systolic and diastolic function. In FIG. 12a, function indices of myocardial anatomy were assessed including thickness of the left ventricular free wall (LVFW) and posterior wall (LVPW) at the end of cardiac function (systole) and relaxation (diastole). Measurements are of wall thickness in mm. Within FIG. 12b, cardiac function was assessed using echocardiography. Indices include stroke volume (Stroke V) which is the volume expelled from the left ventricular upon each cardiac contraction (μL), ejection fraction (EF) which is the percentage of the left ventricular volume expelled with each cardiac contraction (%), fractional shortening (FS) which is the proportion of diastolic dimension lost in systole (%), and cardiac output (CO) which is the total cardiac output per minute (stroke volume×heart rate)(ml/min). Data represent the vehicle (water with 5% (v/v) DMSO and 1% (v/v) 1N HCL) control (solid border) and maraviroc treated mice (non-solid border).

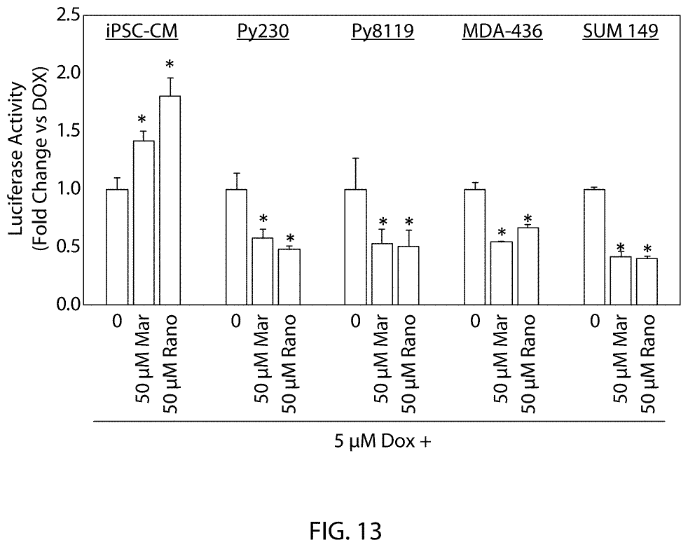

FIG. 13 shows that “dual function” compounds provide cardioprotection and enhance breast cancer cell killing in cultured cells. iPSC were differentiated into cardiomyocytes (CM) by standard protocol (blue, IPSC-CM) or the series of breast cancer cell lines (BCa), were treated with either 5 μM Dox alone or added with maraviroc (Mar, 50 μM) or Ranolazine dihydrochloride (Rano 50 μM). CellTiter-Glo® luminescent cell viability assays (Promega, USA) show improved survival of iPSC-CM and reduced survival of BCa cells in presence of dual function compounds (data are mean+SD, P<0.01).

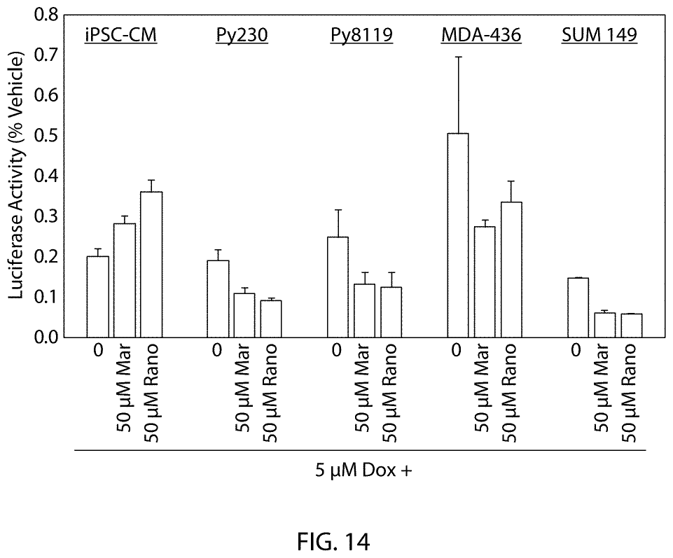

FIG. 14 shows a chart showing luciferase activity as a percent of vehicle for various cells treated with DOX alone or with either maravoric or ranolazine. iPSC were differentiated into cardiomyocytes (CM) by standard protocol (blue, IPSC-CM) or the series of breast cancer cell lines (BCa), were treated with either 5 μM Dox alone or added with maraviroc (Mar, 50 μM) or Ranolazine dihydrochloride (Rano 50 μM). CellTiter-Glo® luminescent cell viability assays (Promega, USA) show improved survival of iPSC-CM and reduced survival of BCa cells in presence of dual function compounds (data are mean+SD, P<0.01).

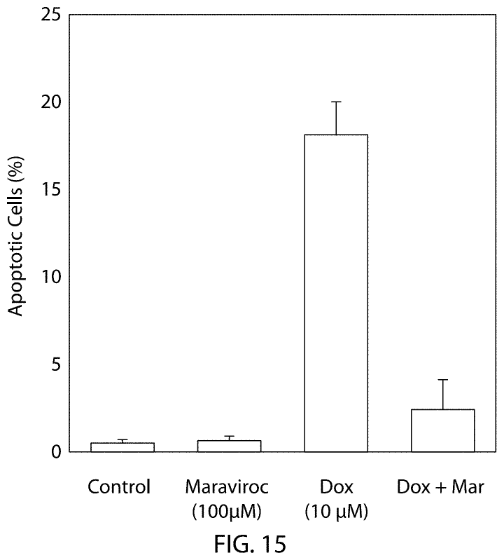

FIG. 15 shows a chart showing that CCR5 inhibitors (i.e. maraviroc) reduced the proportion of DOX-induced apoptotic cell death from 17% to 3% (P<0.05). Isolated canine cardiac myocytes were pretreated with CCR5i (maraviroc, 100 μM) then exposed to DOX (10 μM) for 24 hours. The data suggests that CCR5 activation on myocytes promotes myocardial damage and that CCR5i have direct cardioprotective effects as well as secondary effects related to inflammation.

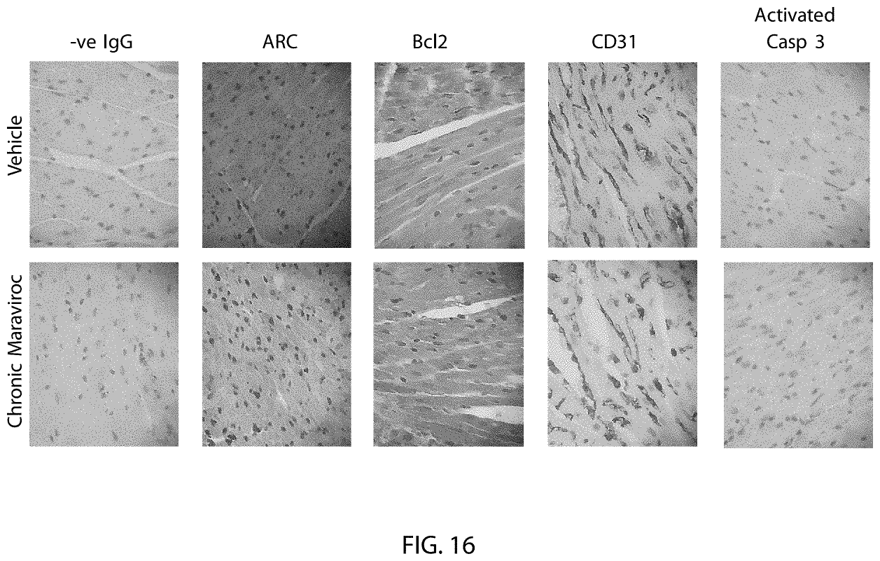

FIG. 16 depicts shows photomicrographs of myocardium stained for common apoptotic mediators in mice treated chronically with maraviroc (16 mg/kg, twice daily gavage; 9 months) or vehicle control (n=3 per group). After 9 months hearts were excised and fixed overnight in paraformaldehyde. Fixed tissues were embedded in paraffin blocks and sectioned (5 mm) in preparation for immunostaining. After deparaffinization and rehydration, antigen retrieval was performed in Tris buffer (pH 9) on the resulting sections. After blocking sections were incubated with an antibodies to assess vascularity (CD31) and myocardial health (ARC, Bcl2, Activated Caspase 3). Antibody binding was visualized using an HRP-conjugated second antibody and Nova red substrate. Haematoxylin was used for nuclear visualization. The results suggest that chronic consumption of CCR5 antagonists did not change the vascularity of the myocardium nor did chronic maraviroc consumption significantly impact cardiac myocyte health.

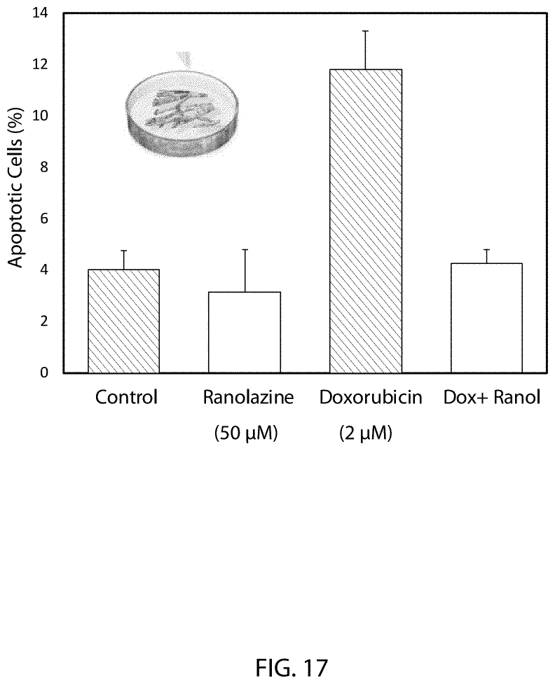

FIG. 17 shows that ranolazine dihydrochloride reduces DOX-induced cardiac toxicity. Isolated canine cardiac myocytes were pretreated with ranolazine dihydrochloride (50 μM) then exposed to DOX (2 μM) for 24 hours and the levels of apoptosis was determined (n=3).

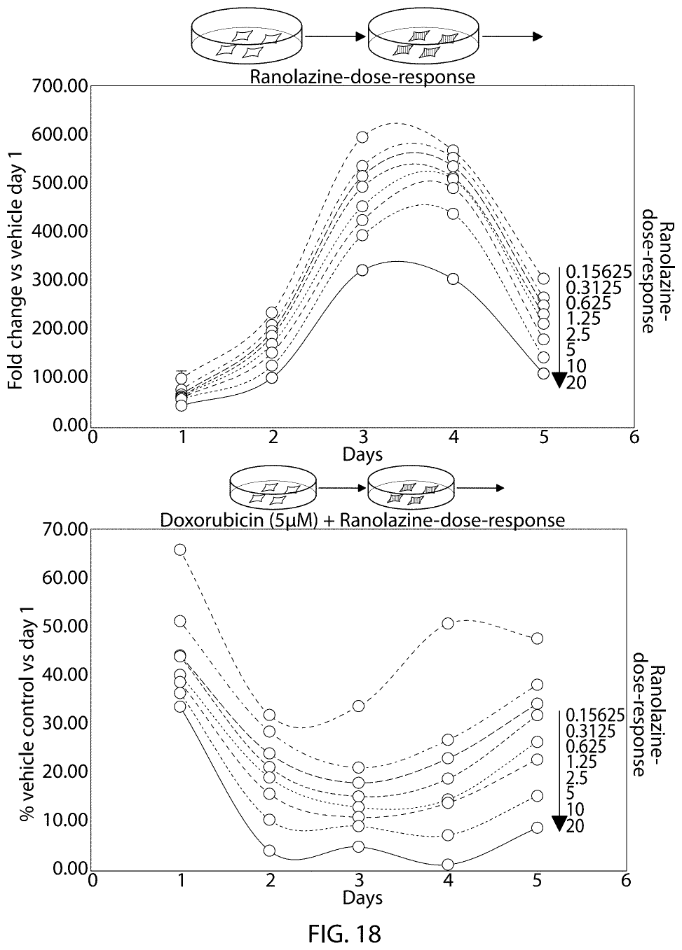

FIG. 18 shows that “dual function” compounds provide cardioprotection and enhance breast cancer cell killing in a dose dependent manner in cultured cells. The breast cancer cell line Py8119 was treated with either increasing doses of Ranolazine (0.156 um to 20 um) or with the addition of doxorubicin. (5 uM Dox). Cell proliferation was established by cell number using methylene blue.

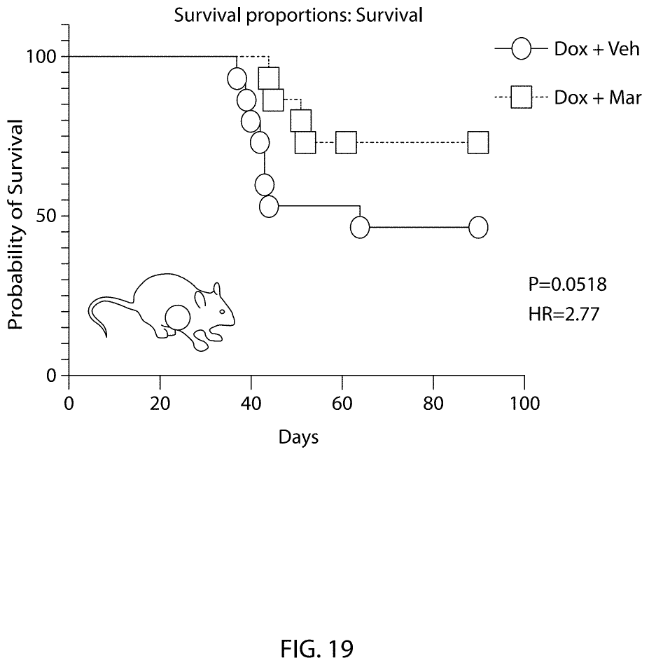

FIG. 19 shows that co-administration of CCR5 inhibitors prevents doxorubicin induced mortality. Mice (n=15 per group) were treated with either doxorubicin with vehicle control (8x 3 mg/kg over 2 weeks; total 24 mg/kg) or doxorubicin with maraviroc (16 mg/kg, twice daily gavage). Survival was documented over the first 90 days after the beginning of treatment. Mice treated with maraviroc had greater survival (10/15 mice) compared to vehicle treated group (7/15 mice). Maraviroc co-treatment enhanced survival 2.77 fold over the vehicle in DOX treated mice.

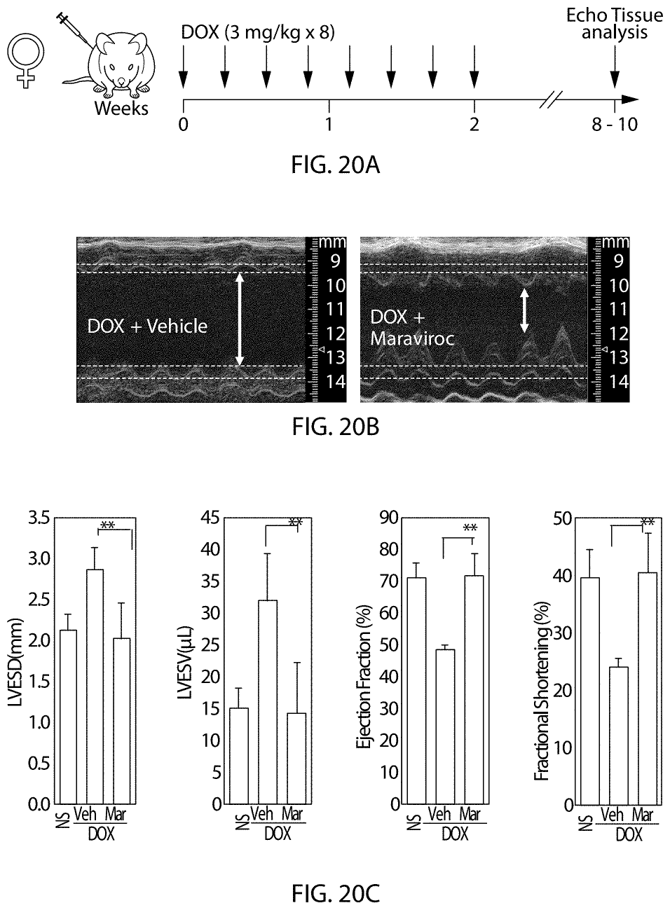

FIGS. 20a to 20c show that co-administration of CCR5 inhibitors prevents doxorubicin induced cardiac dysfunction. FIG. 20a shows a schematic representation of the study protocol in which mice (n=15 per group) were treated with either doxorubicin (8×3 mg/kg over 2 weeks; total 24 mg/kg) and vehicle control or doxorubicin with maraviroc (16 mg/kg, twice daily gavage). FIG. 20b shows echocardiography's for cardiac function in mice conducted 8 weeks after the final dose of doxorubicin using ultrasound imaging. A representative example of the echocardiogram is shown. FIG. 20c shows cardiac function assessed using echocardiography (shown as mean+SEM). Indices include Left Ventricular End Systolic Dimension (LVESD) and Volume (LVESV), which are the diameter and volume of the left ventricle at the end of systole respectively, ejection fraction (EF) which is the percentage of the left ventricular volume expelled with each cardiac contraction (%), and fractional shortening (FS) which is the proportion of diastolic dimension lost in systole (%).

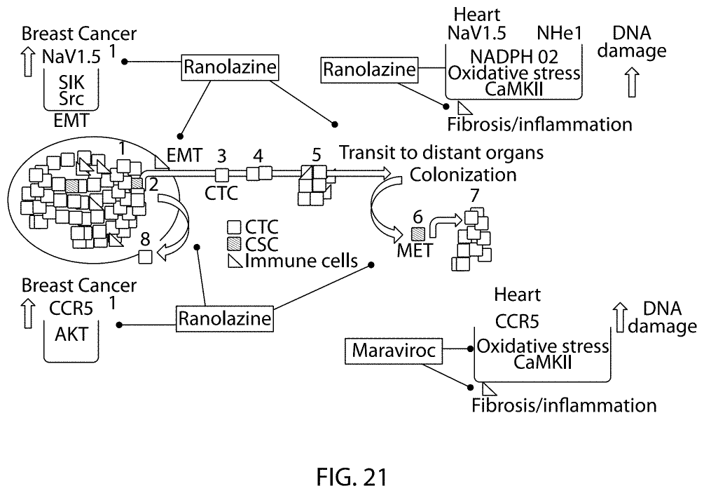

FIG. 21 shows a hypothetical model by which dual purpose agents provide cardioprotection and enhanced cancer cell killing. Within step (A), breast cancer induces NaV1.5 and in turn EMT—which is inhibited by Ranazoline. Step (B) shows a schematic of tumor progression via EMT to metastasis. Step (C) shows CCR5 induced in cancer and in the heart by DNA damaging agents.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1-90. (canceled)

91. A method for administering a chemotherapeutic agent to a patient in need thereof comprising administering an effective amount of a CCR5 antagonist contemporaneously with an effective amount of a chemotherapeutic agent.

92. The method according to claim 91, wherein the chemotherapeutic agent is a DNA damage inducing agent.

93. The method according to claim 91, wherein the chemotherapeutic agent is selected from: an anthracycline; a Her2 inhibitor; an immune checkpoint inhibitor; and a combination of two or more thereof.

94. The method according to claim 93, wherein the chemotherapeutic agent is an anthracycline selected from: daunorubicin; doxorubicin; epirubicin; idarubicin; valrubicin; mitoxantrone; and a combination of two or more thereof.

95. The method according to claim 93, wherein the chemotherapeutic agent is a Her2 inhibitor selected from: trastuzumab; lapatinib; neratinib; pertuzumab; dacomitinib; and a combination of two or more thereof.

96. The method according to claim 93, wherein the chemotherapeutic agent is an immune checkpoint inhibitor comprising a CTLA4/PD-1/PD-L1 selected from: cemiplimab; nivolumab; pembrolizumab; avelumab; durvalumab; atezolizumab; ipilimumab; and a combination of two or more thereof.

97. The method according to any claim 91, wherein the CCR5 antagonist is selected from: a small molecule; an immunotherapy; siRNA/CRISPR; a gene therapy; and a combination of two or more thereof.

98. The method according to claim 97, wherein the small molecule is selected from: maraviroc; vicriviroc; and a combination thereof.

99. The method according to claim 91, wherein the effective amount of the CCR5 antagonist is from about 1 mg/kg/day to about 200 mg/kg/day, optionally from about 10 mg/kg/day to about 190 mg/kg/day, or about 20 mg/kg/day to about 180 mg/kg/day, or about 30 mg/kg/day to about 170 mg/kg/day, or about 40 mg/kg/day to about 160 mg/kg/day, or about 50 mg/kg/day to about 150 mg/kg/day, or about 60 mg/kg/day to about 140 mg/kg/day, or about 70 mg/kg/day to about 130 mg/kg/day, or about 80 mg/kg/day to about 120 mg/kg/day, or about 90 mg/kg/day to about 110 mg/kg/day, or about 100 mg/kg/day.

100. A method of:
treating, preventing, or ameliorating a symptom associated with cardiotoxicity resulting from the administration of a chemotherapeutic agent;
enhancing cardiac function in a patient in need thereof;
increasing survival rate or extending survival time in a patient undergoing treatment with a chemotherapeutic agent; and/or
reducing the effective dose of a chemotherapeutic agent in a patient in need thereof;
the method comprising:
administering an effective amount of a CCR5 antagonist contemporaneously with an effective amount of a chemotherapeutic agent.

101. The method according to claim 100, wherein the chemotherapeutic agent is a DNA damage inducing agent.

102. The method according to claim 100, wherein the DNA damage inducing agent is selected from: an anthracycline selected from: daunorubicin; doxorubicin; epirubicin; idarubicin; valrubicin; mitoxantrone; and a combination of two or more thereof; a Her2 inhibitor selected from: trastuzumab; lapatinib; neratinib; pertuzumab; dacomitinib; and a combination of two or more thereof; an immune checkpoint inhibitor comprising a CTLA4/PD-1/PD-L1 selected from: cemiplimab; nivolumab; pembrolizumab; avelumab; durvalumab; atezolizumab; ipilimumab; and a combination of two or more thereof; and a combination of two or more thereof.

103. The method according to claim 10, wherein the CCR5 antagonist is selected from: a small molecule selected from: maraviroc; vicriviroc; and a combination thereof; an immunotherapy; siRNA/CRISPR; a gene therapy; and a combination of two or more thereof.

104. The method according to claim 100, wherein the CCR5 antagonist is administered prior to the chemotherapeutic agent.

105. The method according to claim 100, further comprising the step of administering an additional dose of a CCR5 antagonist following administration of the chemotherapeutic agent.

106. The method according to any claim 100, further comprising radiation therapy.

107. A composition comprising:
an effective amount of a chemotherapeutic agent comprising a DNA damage inducing agent;
an effective amount of a CCR5 antagonist; and
a pharmaceutically acceptable carrier.

108. The composition according to claim 107, wherein the DNA damage inducing agent is selected from: an anthracycline selected from: daunorubicin; doxorubicin; epirubicin; idarubicin; valrubicin; mitoxantrone; and a combination of two or more thereof; a Her2 inhibitor selected from: trastuzumab; lapatinib; neratinib; pertuzumab; dacomitinib; and a combination of two or more thereof; an immune checkpoint inhibitor comprising a CTLA4/PD-1/PD-L1 selected from: cemiplimab; nivolumab; pembrolizumab; avelumab; durvalumab; atezolizumab; ipilimumab; and a combination of two or more thereof.

109. A kit for reducing cardiotoxicity associated with chemotherapy comprising:
a CCR5 antagonist;
a chemotherapeutic agent; and
instructions for the administration of each.

110. The kit according to claim 109, wherein the chemotherapeutic agent is a DNA damage inducing agent selected from: an anthracycline selected from: daunorubicin; doxorubicin; epirubicin; idarubicin; valrubicin; mitoxantrone; and a combination of two or more thereof; a Her2 inhibitor selected from: trastuzumab; lapatinib; neratinib; pertuzumab; dacomitinib; and a combination of two or more thereof; an immune checkpoint inhibitor comprising a CTLA4/PD-1/PD-L1 selected from: cemiplimab; nivolumab; pembrolizumab; avelumab; durvalumab; atezolizumab; ipilimumab; and a combination of two or more thereof.
